(19) United States
(12) Patent Application Publication
(10) Pub. No.: US 2008/0249331 A1
(43) Pub. Date: Oct. 9, 2008

(54) Vinyl Acetate Catalyst and Support

(75) Inventors: Robert Edward Allan, Berkshire (GB); John William Couves, Buckinghamshire (GB); George Frederick Salem, Aurora, IL (US); Bruce Leo Williams, East Yorkshire (GB)

Correspondence Address: NIXON & VANDERHYE, PC, 901 NORTH GLEBE ROAD, 11TH FLOOR, ARLINGTON, VA, 22203, US

(73) Assignee: BP Chemicals Limited

(21) Appl. No.: 11/922,244

(22) PCT Filed: Jun. 8, 2006
(86) PCT No.: PCT/GB2006/002100

§ 371 Date: Dec. 14, 2007

Related U.S. Application Data
(60) Provisional application No. 60/693,428, filed on Jun. 24, 2005.

(51) Int. Cl. C07C 67/055; B01J 21/12

(52) U.S. Cl. 560/245; 502/243; 502/250; 502/262

(57) ABSTRACT

A microspheroidal support for the manufacture of a vinyl acetate catalyst which support comprises substantially inert microspheroidal particles of a mixture of silica and 0.5 to 5 wt % (based on the total weight of the support) of aluminium oxide. A vinyl acetate catalyst comprising the microspheroidal support, palladium, at least one metal, M, selected from the group consisting of gold, cerium, copper and mixtures thereof and at least one metal. A, selected from the group consisting of Group I, Group II, lanthanide and transition metal promoters.

The present invention relates to a catalyst and a catalyst support useful in the manufacture of vinyl acetate.

Conventionally, vinyl acetate monomer is produced in the gas phase by reacting ethylene, acetic acid and oxygen in the presence of a supported catalyst in a fixed bed reactor. In this type of reactor, a support material such as silica or alumina is impregnated with a catalytic metal such as palladium in combination with gold and an alkali metal salt, typically in the form of an acetate. A requirement of a fixed bed reactor process is that the supported catalyst is formed into relatively large structural shapes such as balls and may be 2 to 5 mm in diameter or more.

Recently vinyl acetate monomer has been produced using a fluid-bed process in which ethylene, acetic acid and oxygen are contacted continuously with a fluidised bed of small supported catalyst particles. Typically, these supported catalyst particles comprise palladium and gold species. Benefits of a fluidised bed vinyl acetate process include a simpler fluid bed reactor design than a multi-tubular fixed bed reactor and higher production rates may be achieved because higher oxygen levels safely may be fed into a fluid-bed reactor without producing a flammable mixture. U.S. Pat. Nos. 5,591,688, 5,665,667, and 5,710,318, are directed to the production of fluid bed vinyl acetate catalysts, or a fluid bed process for the manufacture of vinyl acetate. The fluidised bed reaction may be carried out at a temperature in the range 100 to 250° C. and at a pressure of 50 to 200 psig. The reaction produces vinyl acetate product and as a by-product water.

There is a continuing need for vinyl acetate catalysts which have more advantageous activity characteristics and/or increased catalyst life. The catalyst and catalyst support of this invention show improved hydrothermal stability.

Accordingly, the present invention provides a microspheroidal support for the manufacture of a vinyl acetate catalyst which support consists of substantially inert microspheroidal particles of a mixture of silica and 0.5 to 5 wt % (based on the total weight of the support) of aluminium oxide.

The aluminium oxide may be alumina, such as fumed alumina.

The microspheroidal support may be used in the preparation of catalysts to be employed in either a fixed bed or a fluid bed vinyl acetate process, preferably, a fluid bed process.

By microspheroidal is meant throughout this specification that at least 90% of the silica and/or support particles have a mean diameter of less than 300 microns.

In one embodiment of the present invention the microspheroidal support may be prepared by a process which comprises the steps:

(i) impregnating substantially inert pre-formed microspheroidal particles of silica with a solution of an aluminium salt;

(ii) drying the impregnated particles to form a dried solid material;

(iii) calcining the dried solid material to form the substantially inert microspheroidal support

wherein the substantially inert microspheoidal support comprises 0.5 to 5 wt % (based on the total weight of the support) of aluminium in its oxide form.

The pre-formed microspheroidal silica particles are impregnated with a solution of an aluminium salt. The aluminium salt species should be completely dissolved in a suitable solvent medium, preferably water. Preferably, impregnation with the soluble aluminium species is conducted at ambient temperatures such as 10° to 40° C., usually 20° to 30° C. A preferable method to impregnate the aluminium solution is an incipient wetness technique in which an amount of salt solution measured to fill the pores of the silica particles without excess solution is used. Suitable aluminium salts include aluminium nitrate and aluminium acetate.

The quantity of the soluble aluminium salt species used in the impregnation step is sufficient so as to provide 0.5 to 5 wt %, for example 1 to 5 wt % (based on the total weight of the support) of aluminium in its oxide form in the final support.

The impregnated silica particles are dried to form a dried solid material. The drying may be carried out at any suitable temperature but is typically in the range 40° to 100° C., such as 50° to 80° C. This dried solid material is then calcined to form a substantially inert microspheroidal support of the present invention.

Calcination is preferably performed by heating to a temperature of from 200° to 750° C., preferably 300° to 660° C., suitably in air or oxygen.

Where the support is to be used in a fixed bed process for the manufacture of vinyl acetate, the support suitably has a pore volume of from 0.2 to 3.5 ml per gram of support and suitably has a (BET) surface area of from 5 to 800 m² per gram of support.

Preferably, the preformed microspheroidal silica particles are prepared by forming an aqueous mixture of a silica sol and a particulate silica, followed by spray drying and calcining to form microspheroidal silica particles.

Preferably, the aqueous mixture of the silica sol and the particulate silica is formed from between 20 wt % to less than 100 wt % of silica sol with 80 wt % to greater than 0 wt % of solid particulate silica. Preferably, at least 25 wt %, preferably at least 50 wt % silica sol is mixed with the particulate silica.

Sufficient particulate silica is added to the silica sol to obtain a desired pore volume in the resulting support particle. Preferably, 10 wt % to 50 wt % of the particulate silica is mixed with the silica sol.

The aqueous mixture of the silica sol and particulate silica is spray dried at an elevated temperature in the range 125 to 280° C., preferably 130 to 240° C. The spray dried support is then calcined, preferably at a temperature in the range 550 to 700° C., such as 600 to 660° C. to form the microspheroidal silica support particles.

In an alternative embodiment, a substantially inert microspheroidal support of the present invention, may be prepared by incorporating a particulate aluminum oxide, such as fumed alumina, into the preparation of a microspheroidal silica. The substantially inert microspheroidal support so prepared is suitable for use in the fluid bed manufacture of vinyl acetate.

Accordingly, the present invention provides a process for the preparation of a substantially inert microspheroidal support which process comprises the steps:

(a) mixing less than 100% to 20 wt % of an aqueous sol comprising substantially inert microspheroidal silica particles with greater than 0% to 80 wt % of solid substantially inert particulate silica material to form a first aqueous mixture;

(b) mixing the aqueous mixture with 0.5 to 5 wt % (based on the total weight of the support) of aluminium oxide to form a second aqueous mixture;

(c) spray drying the second aqueous mixture to form dried microspheroidal particles;

(d) calcining the dried microspheroidal particles to form the substantially inert microspheroidal support.

The aqueous mixture of the silica sol and the particulate silica is formed from between 20 wt % to less than 100 wt % of silica sol with 80 wt % to greater than 0 wt % of solid particulate silica. Preferably, at least 10 wt %, preferably at least 50 wt % silica sol is mixed with the particulate silica.

Sufficient particulate silica is added to the silica sol to obtain a desired pore volume in the resulting support particle. Preferably, 10 wt % to 50 wt % of the particulate silica is mixed with the silica sol. To this aqueous mixture is added 0.5 to 5 wt % of aluminium oxide.

The aqueous mixture comprising the aluminium oxide is then spray dried at an elevated temperature of between 115° to 280° C., preferably 130° to 240° C. to form microspheroidal particles which are then calcined, suitably in air or oxygen and preferably at a temperature of between 550° to 700 C., such as 600° to 660° C. to form the substantially inert microspheroidal support of the present invention.

At least 90% of the substantially inert microspheroidal support particles of the present invention have mean particle diameters of less than 300 microns. Suitably 50% of the particles are less than 105 microns, preferably at least 75% of the particles are less than 105 microns and more preferably at least 85% are less than 105 microns. In a typical support useful in this invention, there may be less than 1 to 5% of particles more than 105 microns. Further, typically, less than 50% are less than 44 microns and preferably less than 35% are less than 44 microns. A typical support may contain about 25 to 30% of the particles less than 44 microns. A typical support useful in this invention has at least 50% of the particles with mean diameters between 44 and 88 microns. Persons skilled in the art will recognize that particles sizes of 44, 88, 105 and 300 microns are arbitrary measures in that they are based on standard sieve sizes. Particle sizes and particle size distributions may be measured by an automated laser device such as a Microtrac 100.

The substantially inert microspheroidal support particles are sufficiently porous to permit gaseous reactants to diffuse into the particle and contact catalytic sites incorporated within the particle. Thus, the pore volume should be high enough to permit gaseous diffusion. However, a particle with an exceedingly high pore volume typically will not have sufficient attrition resistance or will not have sufficient surface area for catalytic activity. A typically sufficient microspheroidal particle has a pore volume (measured by nitrogen sorption) between about 0.2 and 0.7 cc/gram. A preferable particle has a pore volume between about 0.3 and 0.65 cc/g and more preferably between about 0.4 and 0.55 cc/g.

Surface areas (measured by BET) for microspheroidal particles with mean diameters and pore volumes useful in this invention typically are above about 50 m²/g and may range up to about 200 m²/g. A typical measured surface area is about 60 to about 125 m²/g.

A suitable particulate silica for use in all of the embodiments of this invention is a fumed silica such as Aerosil® (DeGussa Chemical Company). A typical silica particulate material has a high surface area (such as about 200 m²/g) with essentially no micropores and typically are aggregates (with mean diameters of several hundred nanometres) of individual particles with average diameters of about 10 nm (such as above 7 nm). Preferably, the silica is sodium free.

Suitably, the silica sol useful in all the embodiments of this invention contains silica particles in the sol which typically have a mean diameter of at least 20 nm such as up to about 100 nm or more. Preferable sols contain silica particles having a mean diameter of about 40 to 80 nm. Suitable silica sols are those such as Nalco silica sol 1060 (Nalco Chemical Company).

Advantageously, the substantially inert microspheroidal, supports of the present invention are highly stable under conditions of heat, water, pressure and/or the presence of alkali metal salts. Such conditions are typically present in the manufacture of vinyl acetate and, in particular are present in the fluid bed manufacture of vinyl acetate. Thus, the substantially inert microspheroidal supports of the present invention are suitable for use in the manufacture of vinyl acetate catalysts.

Thus, the present invention further provides for a vinyl acetate catalyst which catalyst comprises palladium, at least one metal M selected from gold, cerium, copper and mixtures thereof and at least one metal, A selected from Group I, Group II, lanthanide and transition metals promoters supported on a substantially inert microspheroidal support as hereinabove described or prepared.

The present invention yet further provides a process for the manufacture of a fluid bed vinyl acetate catalyst of formula Pd-M-A where M is at least one metal selected from gold, cerium, copper and mixtures thereof and A is at least one metal selected from Group I, Group II, lanthanide and transition metals promoters which process comprises:

(i) impregnating a substantially inert microspheroidal support of the present invention with (a) a solution comprising a metal salt of palladium, M and a salt of at least one metal A selected from Group I, Group II, lanthanide and transition metals promoters or (b) a solution comprising a metal salt of palladium and M and either a solution or a solid salt of at least one metal A selected from Group I, Group II, lanthanide and transition metals promoters; and

(ii) drying the impregnated microspheroidal support to form the catalyst.

The microspheroidal support is impregnated with at least one compound of palladium such that the catalyst typically contains at least about 0.1%, preferably at least 0.2 wt % palladium to about 5 wt % and preferably up to 4 wt % palladium.

The impregnation of the soluble metal salts may be conducted by any known procedure. Preferably, the microspheroidal support is impregnated by the incipient wetness technique in which an amount of salt solution(s) measured to fill the pores of the support without excess solution is used. Typically in this technique the support is contacted with a solution of the salts to be impregnated in an amount which is from 60 to 120% of the pore volume of the support particles, preferably from 70 to 100% of the pore volume. Suitable solvents may be water, carboxylic acids such as acetic acid, benzene, toluene, alcohols such as methanol or ethanol, nitrites such as acetonitrile or benzonitrile, tetrahydrofuran or chlorinated solvents such as dichloromethane. Preferably, the solvent is water and/or acetic acid. Suitably, the support is impregnated with palladium acetate, sulphate, nitrate, chloride or halogen-containing palladium compounds such as H₂PdCl₄, which is sometimes also represented as [PdCl₂]2HCl, and Group I or Group II salts thereof such as Na₂PdCl₄ and K₂PdCl₄. A preferred water soluble compound is Na₂PdCl₄. A preferred acetic acid-soluble palladium compound is palladium acetate. The palladium compounds may be prepared in situ from suitable reagents.

The catalyst also comprises other metals such as gold, cerium, copper and mixtures thereof, preferably gold. These metals may be used in an amount of 0.1 to 10% by weight of each metal present in the finished catalyst composition. Typically, the weight percent of gold is at least about 0.1 wt %, preferably, at least 0.2 wt % gold to about 3 wt % and preferably up to 2 wt % gold., Suitable gold compounds which may be used include gold chloride, dimethyl gold acetate, barium acetoaurate, gold acetate, tetrachloroauric acid (HAuCl₄, sometimes represented as AuCl₃.HCl) and Group I and Group II salts of tetrachloroauric acid such as NaAuCl₄ and KAuCl₄. Preferably, the gold compound is HAuCl₄. The gold compounds may be prepared in situ from suitable reagents.

Suitably, the support is impregnated with a solution comprising palladium and gold compounds.

The support may be simultaneously impregnated with a solution of palladium, M and A or may be impregnated with a solution of palladium and M and subsequently impregnated with a solution or solid salt of A.

The impregnated support may be optionally subjected to a reduction step.

Preferably, the impregnated metal species incorporated within the support such as palladium and gold species are reduced by contact with a suitable reducing agent. This reduction will transform the impregnated palladium species to catalytically active zero valance forms of palladium such as crystallites and/or palladium/gold alloys. Typical reducing agents include hydrogen, hydrides, alkenes and hydrazine. Preferably, hydrazine (most preferably in an aqueous solution) is used to reduce the metal species. Preferably the solution of hydrazine is an aqueous solution of hydrazine that has not been rendered alkaline by an alkali metal hydroxide. Most preferably the solution of hydrazine is an aqueous solution of hydrazine in the absence of any other added components.

Preferably, the concentration of hydrazine in the aqueous solution is 1 to 20 wt %, such as 3 to 20 wt %, for example 4 to 20 wt %.

Reduction with aqueous hydrazine after impregnation is preferable.

Preferably, the impregnated support is added to a solution of the hydrazine rather than the addition of the hydrazine solution to the impregnated support.

Typically an excess of reducing agent is used to complete the reduction.

Preferably, impregnated and reduced catalyst support particles are washed with a suitable solvent such as water to remove excess reducing agent as well as undesired anions such as halides. Washing may be performed several times with portions of the wash solvent until the desired level of contaminants is reached. Typically, the washed particles are dried slowly at an elevated temperature such as 40 to 80° C.

Where the impregnated support is to be treated with an aqueous solution of hydrazine, it is preferably dried prior to the treatment with hydrazine at a temperature in the range 50 to 200° C., preferably 100 to 150° C.

Dry gas such as air, nitrogen, at room temperature to 200° C. may be passed over and/or through the impregnated support during drying.

In addition to palladium and the metal selected from gold, copper and cerium the microspheroidal support is impregnated with one or more salts of Group I, Group II, lanthanide and transition metals promoters preferably cadmium, barium, potassium, sodium, manganese, antimony, lanthanum or mixtures thereof, which are present in the finished catalyst composition as salts, typically acetates. Generally, potassium will be present. Suitable salts of these compounds are acetates but any soluble salt may be used. These promoters may be used in an amount of 0.1 to 15%, preferably 3 to 9%, by weight of each promoter salt present in the finished catalyst composition. The promoter salts may be impregnated by blending the support with solid salts of the promoter metal in the presence of limited amount of solvent.

In one embodiment, the one or more salts of Group I, Group II, lanthanide and transition metals is separately impregnated onto the support, preferably subsequently to the impregnation of the solution comprising the salts of palladium and the M element onto the support and the reduction thereof with a suitable reducing agent.

Preferably, after impregnation of the support with one or more salts of Group I, Group II, lanthanide and transition metals it is dried at a temperature in the range from 40° C. to 150° C.

In a preferred embodiment of the catalyst preparation, impregnation of the support with a solution of palladium and gold compounds is followed by drying of the impregnated support, the dried impregnated support is then added to an aqueous solution of hydrazine. Following the reduction with hydrazine, either (i) a solid salt of potassium is added to the solid support material and then mixed or (ii) the reduced solid support material is impregnated with a solution of a potassium salt. Subsequent to (i) or (ii) the material is dried to form the finished catalyst.

A typical catalyst useful in a fluidised bed process may have the following particle size distribution:

| | | | |
|---|---|---|---|
| 0 to 20 | microns | 0-30 | wt % |
| 20 to 44 | microns | 0-60 | wt % |
| 44 to 88 | microns | 10-80 | wt % |
| 88 to 106 | microns | 0-80 | wt % |
| >106 | microns | 0-40 | wt % |
| >300 | microns | 0-5 | wt % |

The catalysts comprising the supports of the present invention may be used in a fixed bed or a fluid bed process, preferably a fluid bed process for the reaction of ethylene and acetic acid with a molecular oxygen-containing gas, such as oxygen to produce vinyl acetate. The reaction temperature may suitably be in the range 100 to 250° C., preferably in the range 130 to 190° C. The reaction pressure is suitably in the range 50 to 200 psig (3 to 14 barg), preferably in the range 75 to 150 psig (5 to 10 barg).

The invention will now be described by reference to the following Examples.

### Support Preparation

### Support A

Pre-formed microspheroidal silica particles were prepared by spray drying a mixture of Nalco (Nalco Chemical Company) silica sol 1060 and Aerosil® 200 silica (DeGussa Chemical Company). In the dried support 80% of the silica came from the sol and 20% of the silica came from the Aerosil. The spray dried microspheres were calcined in air at 640° C. for 4 hours.

### Support 1

Support 1 was prepared by impregnating 5.72 g of Support A with 2.217 g of aluminium nitrate hydrate dissolved in 15 ml of water by an incipient wetness technique. The mixture was stirred and left to stand at ambient temperature for 1 hour. The impregnated solid was then dried overnight at a temperature of 120° C. The dried solid was calcined in air for 4 hours at 300° C. and for a subsequent 4 hours at 640° C. The resulting microspheroidal support contained 5 wt % alumina.

### Support 2

Support 2 was prepared by spray drying a mixture of Nalco (Nalco Chemical Company) silica sol 1060 and Aerosil® 200 silica (DeGussa Chemical Company) and fumed alumina oxide C (Degussa Chemical Company). In the dried support 79.2% of the silica came from the sol and 19.8% of the silica came from the Aerosil and 1% of the support came from the aluminium oxide. The spray dried microspheres were calcined in air at 640° C. for 4 hours.

The resulting microspheroidal support contained 1 wt % alumina.
Support 3 (5 wt % from Fumed Alumina)

Support 3 was prepared by spray drying a mixture of Nalco (Nalco Chemical Company) silica sol 1060 and Aerosil® 200 silica (DeGussa Chemical Company) and fumed alumina oxide C (Degussa Chemical Company). In the dried support 76% of the silica came from the sol and 19% of the silica came from the Aerosil and 5% of the support came from the aluminium oxide. The spray dried microspheres were calcined in air at 640° C. for 4 hours.

The resulting microspheroidal support contained 5 wt % alumina.
Support 4 (2 wt % from Aluminium Nitrate)

Support 4 was prepared by impregnating 52.32 g of Support A with 7.87 g of aluminium nitrate hydrate dissolved in 33.6 g of water by an incipient wetness technique. The mixture was stirred and left to stand at ambient temperature for 1 hour. The impregnated solid was then dried overnight at a temperature of 120° C. The dried solid was calcined in air for 4 hours at 300° C. and for a subsequent 4 hours at 640° C. The resulting microspheroidal support contained 2 wt % alumina.

### SUPPORT TESTING EXAMPLES 1-2 AND COMPARATIVE EXPERIMENT A

A series of autoclave experiments were conducted to demonstrate the change in porosity of a 100% microspheroidal silica support (Support A) and microspheroidal silica supports comprising 5 wt % and 1 wt % alumina (Supports 1 and 2 respectively)

The porosity of a 1.5 g sample of each support was monitored by nitrogen porosimetry. The support sample was then each heated in 15 ml of water in a PTFE lined Parr autoclave (23 ml) for 24 hours at 175° C. after which the porosity was re-monitored. The results of the experiments are shown in FIGS. 1 to 3. The Figs. show the the porosity of each of the supports before and after heating in the autoclave. The less the broadening of the pores in the support, the greater the stability of the support to hydrothermal conditions.

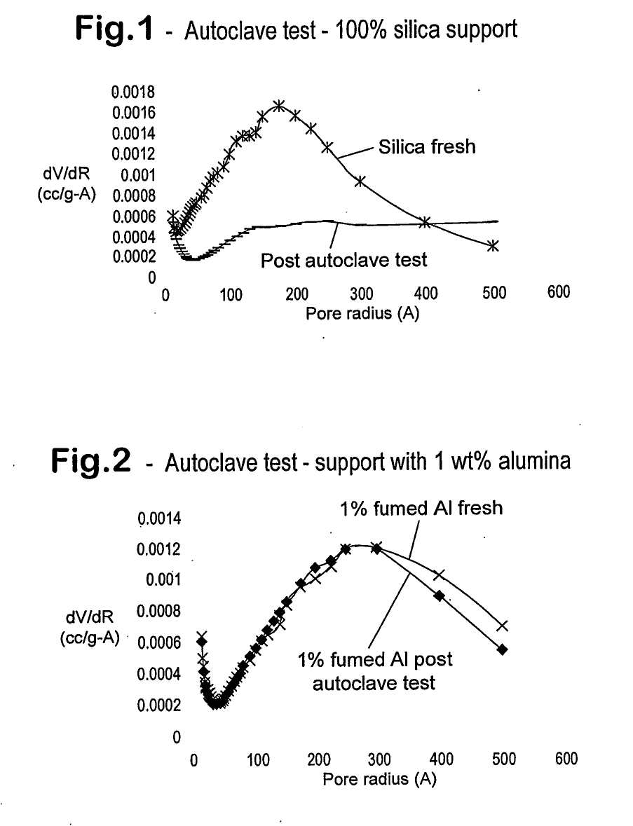

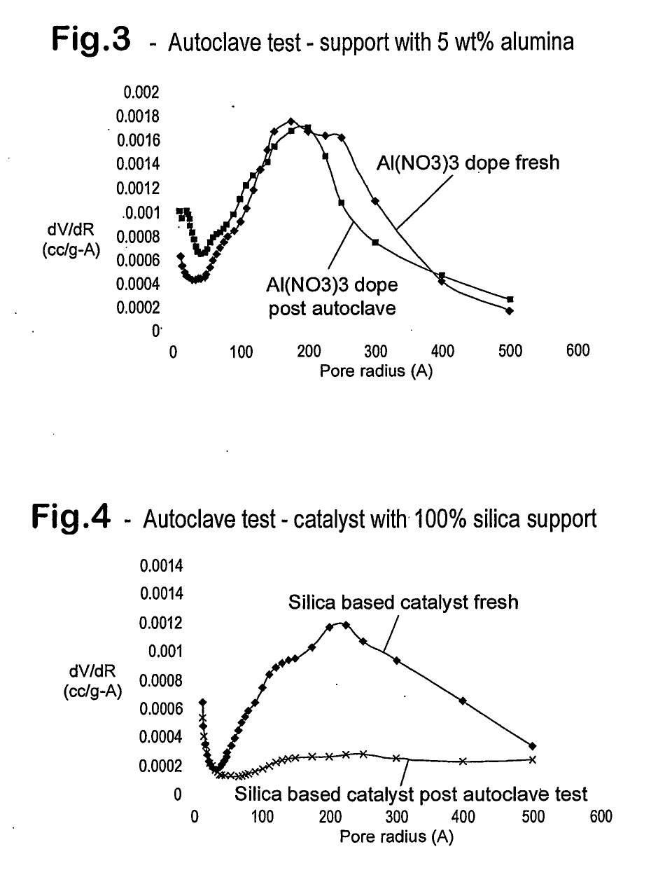

As can be seen from FIG. 1 the 100% silica support has a much reduced porosity after application of the hydrothermal conditions. This is indicated by the loss of porosity of the pores with a radius of less than 500 A. However, from an inspection of FIG. 2 ( 1 wt % alumina) and FIG. 3 (5 wt % alumina) it can be seen that there is very little change in the porosity and pore broadening of the supports of the invention after application of the hydrothermal conditions.

### Catalyst Preparation

Vinyl acetate catalysts comprising palladium, gold and potassium were prepared by impregnating Support A and Support 1 with solutions of palladium and gold, dried overnight at 60° C. The dried solid material was then treated with a liquid reductant, dried overnight at 60° C. The dried solid material was then impregnated with a solution of potassium.

### CATALYST TESTING EXAMPLES 3 AND 4 AND COMPARATIVE EXPERIMENTS B AND C

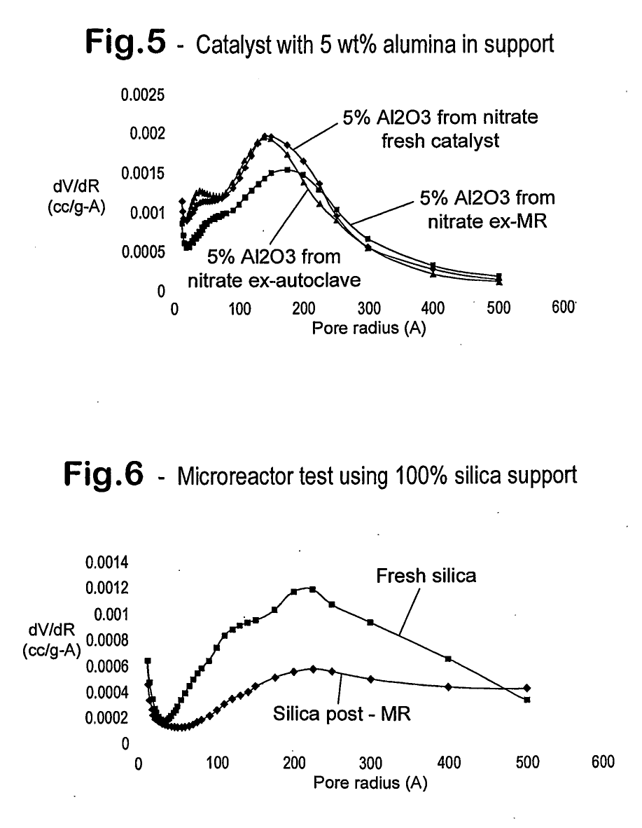

The catalyst samples were tested to determine their stability to hydrothermal conditions using an autoclave test and a microreactor test. In the autoclave experiment the porosity of a 1.5 g sample of each catalyst was monitored by nitrogen porosimetry. The catalyst sample was then each heated in 15 ml of water in a PTFE lined Parr autoclave (23 ml) for 24 hours at 175° C. after which the porosity was re-monitored. The results of the autoclave experiment (Comparative B) for the catalyst prepared from Support A is given in FIG. 4 and the results of the autoclave experiment (Example 3) for the catalyst prepared from Support 1 i.e. a support according to the present invention is given in FIG. 5.

In the microreactor experiments, the catalyst sample was fluidized in a 40 cc microreactor for 6 hours at 150° C. under a flow of 10% water and 90% nitrogen at a pressure of 8 barg. The results of the of the microreactor experiment (Comparative C) for the catalyst prepared from Support A is given in FIG. 6 and the results of the autoclave experiment (Example 4) for the catalyst prepared from Support 1 i.e. a support according to the present invention is given in FIG. 5. The results show that the support made according to the invention (Support 1) retains significantly more of its pore volume than that of Support A

### PREPARATION OF VINYL ACETATE EXAMPLES 5 TO 8 AND COMPARATIVE EXPERIMENTS D AND E

A series of experiments were conducted to prepare vinyl acetate using catalysts prepared from 100% silica supports (Support A) (Comparative Experiments D and E) and catalysts prepared from supports according to the present invention. Examples 5 and 6 employed Support 3, Example 7 employed Support 4 and Example 8 employed Support 1.

2 g of each catalyst was mixed with 28 ml of diluent and charged to a fluid bed microreactor. The reactants were fed to the microreactor at a gas hourly space velocity of 7580 with a composition at the reactor inlet of 7.8 mol % oxygen, 29.4 mol % nitrogen, 10.9 mol % acetic acid, 51.9 mol % ethylene. The gases were delivered from cylinders via mass flow controllers. The acetic acid was delivered via a syringe drive and vaporized prior to entering the reactor. The reaction was carried out at a pressure of 115 psi and at a temperature of 150° C. Analysis of the reactor exit stream was carried out by gas chromatography. The reaction selectivity was calculated based on ethylene conversion to vinyl acetate and carbon dioxide. The calculated selectivities are quoted as an average of the values obtained over the period from 16 to 20 hours on stream. The activities and selectivities of the catalysts are given in Table 1 below. From the results of the experiments the catalysts prepared from supports according to the invention show increased activity compared to those prepared from 100% silica supports

TABLE 1

| | ACTIVITY | SELECTIVITY | O2 CONVERSION |
|---|---|---|---|
| SUPPORT | (gVAM/kg/hr) | (%) | (%) |
| Comparative D | 1159 | 95 | 24 |
| Example 5 | 1596 | 94 | 36 |
| Example 6 | 1400 | 93 | 35 |
| Example 7 | 1458 | 94 | 33 |
| Comparative E | 1161 | 95 | 24 |
| Example 8 | 1540 | 94 | 27 |

1-35. (canceled)

36. A process for the manufacture of a fluid bed vinyl acetate catalyst of formula Pd-M-A where M is at least one metal selected from gold, cerium, copper and mixtures thereof and A is at least one metal selected from Group I, Group II, lanthanide and transition metals promoters which process comprises:
(i) impregnating a support which consists of substantially inert microspheroidal particles of a mixture of silica and 0.5 to 5 wt % (based on the total weight of the support) of aluminium oxide with
(a) a solution comprising a metal salt of palladium, M and a salt of at least one metal A selected from Group I, Group II, lanthanide and transition metals promoters, or
(b) a solution comprising a metal salt of palladium and M and either a solution or a solid salt of at least one metal A selected from Group I, Group II, lanthanide and transition metal promoters; and
(ii) drying the impregnated microspheroidal support to form the catalyst.

37. A process according to claim 36 wherein M is gold.

38. A process according to claim 36 wherein A is a Group I metal.

39. A process according to claim 38 wherein the Group I metal is potassium.

40. A process according to claim 38 wherein the support is (a) impregnated with a solution of palladium and gold compounds (b) the impregnated dried support is then added to an aqueous solution of a reducing agent, (c) subsequent to the reduction with the reducing agent either (i) a solid salt of potassium is added to the support and then mixed or (ii) the reduced solid support material is impregnated with a solution of a potassium salt and (d) subsequent to (i) or (ii) the material is dried to form the finished catalyst.

41. A process according to claim 36 wherein the support comprises 1 to 5 wt % of aluminium oxide.

42. A process according to claim 36 wherein the microspheroidal particles have a pore volume in the range 0.2 to 0.7 cc/g and a surface area in the range 50 to 200 m2/g.

43. A process according to claim 36 wherein at least 90% of the substantially inert microspheroidal support particles have mean particle diameters of less than 300 microns and at least 50% of the particles are less than 105 microns.

44. A process for the manufacture of vinyl acetate which comprises contacting ethylene, acetic acid and a molecular oxygen-containing gas in the presence of a catalyst prepared according to claim 36.

45. A vinyl acetate catalyst comprising palladium, at least one metal M selected from gold, cerium, copper and mixtures thereof and at least one metal A selected from Group I, Group II, lanthanide and transition metals promoters supported on a support which consists of substantially inert micro spheroidal particles of a mixture of silica and 0.5 to 5 wt % (based on the total weight of the support) of aluminium oxide.

46. A catalyst according to claim 45 wherein the support comprises 1 to 5 wt % of aluminium oxide.

47. A catalyst according to claim 46 wherein at least 90% of the substantially inert microspheroidal support particles have mean particle diameters of less than 300 microns and at least 50% of the particles are less than 105 microns.
